(19) United States
(12) Patent Application Publication
(10) Pub. No.: US 2011/0020893 A1
(43) Pub. Date: Jan. 27, 2011

(54) Trapping discrete particles in fluids (For example, filtering for specific virus, molecule, or other identifiable particle in water, blood, air or other fluids)

(76) Inventor: Andrew Bransford Brown, Scottsdale, AZ (US)

Correspondence Address: Andrew B. Brown, B108, 1100 E Lemon Street, Tempe, AZ, 85281, US

(21) Appl. No.: 12/460,880

(22) Filed: Jul. 27, 2009

(51) Int. Cl. C12N 13/00

(52) U.S. Cl. 435/173.3; 435/173.1

(57) ABSTRACT

The invention describes a way to target and trap a specific particle (atom, molecule, bacterium, photon, ion, virus, etc.) that can be extracted from a moving fluid flow. Since every particle has a unique and identifiable shape and/or electromagnetic signature, a trap can be built to target it. The fluid is assumed to be moving and the trap is to be placed in the flow to capture the specific particle. Other particles would not be captured unless specifically targeted. Once trapped, the particle can be returned to the flow or be pulled out, the trap cleaned and replaced to catch a new particle. This can be used to remove specific particles from the fluid flow for mining purposes or to clean the fluid (EG. removing sodium molecules from flowing water or removing a virus from the blood stream).

### BACKGROUND OF THE INVENTION

This invention relates to removing foreign bodies from blood (virus, bacteria, etc.), desalinating water, mining for molecules in fluids, and killing viruses/bacteria in the blood stream without surgery.

### SUMMARY OF THE INVENTION

The invention focuses on identifying the specific electromagnetic frequencies that orient a specific particle to guide the particle into a physical or electromagnetic pocket or sleeve. Once filled, it can be pulled from the fluid flow by mechanical or electromagnetic means and emptied to be refilled. Also included in the invention is identifying frequencies or electromagnetic parabolas that dislodge the electron or kill the particle without damaging surrounding particles or the fluid itself.

What is claimed is:

1. Guiding and trapping a particle (atom, molecule, bacterium, virus, etc.) by one or more of the following methods:
physically or electromagnetically guiding the particle into a physical trap in the inverse of the shape of the particle.
electromagnetically identifying, arranging and guiding the particle into an electromagnetic trap designed to hold that specific particle
removing the electron and capturing only the neutron/proton
electromagnetically modifying a particle to change its structure or render it inert. EG. targeting the nucleus of a virus to kill it using a specific electromagnetic frequency that only that virus responds to.
other possible areas of research: photon, laser, and quantum effects to identify and trap particles.
